(19) United States
(12) Patent Application Publication
(10) Pub. No.: US 2023/0330226 A1
(43) Pub. Date: Oct. 19, 2023

(54) COMPOSITIONS AND METHODS FOR THE PREVENTION OF TYPE I HYPERSENSITIVITY REACTIONS

(71) Applicant: IgGenix, Inc., South San Francisco, CA (US)

(72) Inventor: Roger Thomas, South San Francisco, CA (US)

(21) Appl. No.: 18/134,854

(22) Filed: Apr. 14, 2023

Related U.S. Application Data
(60) Provisional application No. 63/331,601, filed on Apr. 15, 2022.

(51) Int. Cl. A61K 39/395

(52) U.S. Cl. CPC A61K 39/39566; A61K 39/39516; A61K 2039/507

(57) ABSTRACT

The present invention provides compositions and methods for preventing an anaphylactoid hypersensitivity reaction in a subject by providing to that subject a combination of antigen-binding IgG and an anti-IgE antibody.

### TECHNICAL FIELD

The invention relates generally to the fields of medicine, immunology, and allergy, and more particularly to the prevention of anaphylactoid hypersensitivity reactions.

### BACKGROUND

Allergy is estimated to affect 30% of the global population. The prevalence of diagnosed allergies is continually increasing due to numerous factors, but in part by the recognition of new allergens and allergic responses as well as the increased availability of allergy tests.

Allergies are characterized by several conditions caused by hypersensitivity of the immune system to otherwise harmless substances in the environment. In general, an allergic reaction occurs when aspects of the immune system overreact to the presence of a substance (an allergen) that, absent the allergy, would not cause a reaction. Allergies have a negative impact on individuals’ quality of life and can lead to societal and personal economic burdens. As mere exposure to certain allergens can have life-threatening consequences, people suffering from allergies are often required to be hypervigilant and forced to alter their behavior to avoid allergens.

As generally understood, an allergen is a type of antigen that produces an abnormally vigorous immune response in which the immune system combats a perceived threat that would otherwise be harmless. In technical terms, an allergen is an antigen that is capable of stimulating a type I hypersensitivity reaction mediated by immunoglobulin E (IgE) in atopic individuals. Most humans mount significant IgE responses only as a defense against parasitic infections. However, some individuals may respond to common environmental antigens. This hereditary predisposition is called atopy. In atopic individuals, non-parasitic antigens stimulate inappropriate IgE production, leading to type I hypersensitivity.

Emerging research has shown that allergies are not homogenous conditions. Allergy research has shown that many common allergies are more complex than previously thought. For example, patients with peanut allergies may be allergic to one or more proteins found in peanuts as well as one or more epitopes of said proteins. Further, people with a particular allergy can produce different IgE antibodies in response to the same antigen.

Elevated serum IgE levels are associated with allergic disorders, parasitosis and specific immunologic abnormalities. However, epidemiological and mechanistic evidence indicates an association between IgE-mediated immune surveillance and protection from tumor growth. Recent studies reveal a correlation between IgE deficiency and increased malignancy risk. While elevated serum IgE is generally associated with allergic/atopic conditions, very low or absent IgE may hamper anti-tumor surveillance, indicating the importance of a balanced IgE-mediated immune function. Ultra-low IgE may prove to be an unexpected biomarker for cancer risk.

### SUMMARY

The present invention provides compositions and methods for the prevention of a Type I hypersensitivity reaction, colloquially referred to as an allergy, in a subject, especially a subject known to have had such a reaction to an allergen in the past. The constituent part of the invention causing that prevention is balanced with the possibly increased risk for cancer associated with long term systemic low serum IgE levels. The invention is directed to the combination of anti-allergen IgG antibodies with an anti-IgE antibody that inhibits IgE binding to the high affinity FcεRI receptor or removes IgE already bound to it. By combining the two strategies either by coadministration, coformulation or the generation of bi-specific IgG antibodies, total IgE can be lowered to a safe level using an anti-IgE; which in turn will require less further attenuation to prevent an anaphylactic response by the addition of a specific anti-allergen IgG. The invention may be carried out with whole antibodies or fragments thereof.

Embodiments of the invention disclose a composition for allergy treatment comprising an IgG antibody that binds to an epitope on an allergen and an anti-IgE antibody in an amount sufficient to inhibit IgE binding to a high affinity FcεRI receptor but that does not eliminate circulating IgE.

According to an embodiment of the invention, the anti-IgE antibody may be omalizumab. Omalizumab is an IgG that acts by binding and depleting circulating IgE. When dosed at levels for the indicated treatments, it can reduce levels of serum IgE by up to 95%. This indiscriminate lowering of the circulating IgE levels not only has the intended effect of reducing the potential for allergic responses such as asthma attacks and anaphylactic shock but since this is life-long therapy, may expose the patient to increased risk of cancer. The anti-IgE antibody of the invention may reduce circulating IgE by at least 25%, at least 50%, or at least 75%.

Monoclonal IgG antibodies against various allergens can potentially be used to attenuate anaphylactic episodes in allergic patients when exposed to allergens. That success is dependent on having adequate amounts of various IgG antibodies to bind multiple epitopes on one or more antigens. This is further compounded by the fact that allergens such as peanuts have many individual antigens e.g., Ara h 1, 2, 3, 6, 8, 9, etc., each of which may have multiple epitopes. However, significant reductions in activity, as measured in ex vivo assays such as the basophil activation test, can be achieved by blocking only some of a single antigen’s epitopes, even in a complex allergen such as peanut. However, since this blockade may not be complete, it may not be adequate to provide complete protection for the allergic patient in the event of allergen exposure.

Therefore, the combination of the anti-IgE antibody with an anti-allergen IgG may serve to reduce the symptoms of a Type I hypersensitivity reaction, while also not compromising the body’s ability to detect and destroy nascent tumors.

According to varying embodiments of the invention, the IgG antibody and anti-IgE antibody may be contained within one dosage form; alternatively, the IgG antibody and anti-IgE antibody may be provided to the subject in separate dosage forms.

Embodiments of the invention also provide methods for treating a condition in a subject, wherein the composition of the invention is provided to that subject. According to varying embodiments of the invention, the condition may be a hypersensitivity reaction of any type, or it may particularly be a Type I hypersensitivity reaction. The condition may be anaphylaxis due to exposure to a food, animal, or environmental allergen.

### DETAILED DESCRIPTION

The invention discloses compositions and methods for the prevention of a Type I hypersensitivity reaction in a subject. The composition is a combination of an IgG antibody that binds to an epitope on an allergen and an anti-IgE antibody in an amount sufficient to inhibit IgE binding to a high affinity FcεRI receptor but that does not eliminate circulating IgE.

Hypersensitivity reactions occur when the immune system has dysfunctional response disproportionate to the antigen presented to the body. Some are immediate anaphylactic reactions while others more slowly develop into other disease states. The invention can be used to prevent the hypersensitivity reaction wherein a subject or a sample from the subject responds with a hypersensitive, or abnormally sudden and severe, response to the stimulus.

Some foods such as peanuts (a legume), nuts, seafood and shellfish are the cause of serious Type I hypersensitivity reactions in many people. Officially, the United States Food and Drug Administration recognizes eight foods as being common for allergic reactions in a large segment of the sensitive population. These include peanuts, tree nuts, eggs, milk, shellfish, fish, wheat and their 20 derivatives, and soy and their derivatives, as well as sulfites (chemical-based, often found in flavors and colors in foods).

An allergic reaction can be caused by any form of direct contact with the allergen-consuming food or drink one is sensitive to (ingestion), breathing in pollen, perfume or pet dander (inhalation), or brushing a body part against an allergy-causing plant (direct contact). The presently disclosed systems and methods can be used to prevent an allergic response in a subject due to ingestion, inhalation, and/or contact with an allergen or source of allergens.

There are five classes of immunoglobulin: IgA, IgD, IgE, IgG, and IgM. Immunoglobulins are antibodies. Whenever the term “antibody” is used in the disclosure it is intended to mean polyclonal antibodies, monoclonal antibodies, or antigen-binding portions or fragments of any of the foregoing. IgA is mainly present in secretions such as bowel fluid, nasal discharge, and saliva, to prevent bacterial invasion from a mucous membrane. It is also present in breast milk and protects the gastrointestinal tract of newborns from bacterial and viral infection. IgD is present on the surface of B cells and it is reported to play a role in the induction of antibody production and the prevention of respiratory tract infections. IgE is related to Type I hypersensitivity reactions. By binding to mast cells, IgE is believed to be involved in allergies such as pollinosis. IgG is the main antibody in blood and has a powerful ability to bind to bacteria and toxins, and thus it takes on an important role in the biological defense system. It is the only isotype that can pass through the placenta, and IgG transferred from the mother’s body protects a newborn. IgM is constructed of five units of basic Y-shaped structures and is mainly distributed to the blood. Produced first upon pathogen invasion by B cells, IgM has a key role in the initial immune system defense for protecting the body. Disclosures of the invention primarily focus on IgG and IgE antibodies.

An antibody or antigen-binding fragment thereof is capable of binding to a known non-tumor allergen. For example, the specific allergen may include, but is not limited to, a food allergen, a plant allergen, a fungal allergen, an animal allergen, a dust mite allergen, a drug allergen, a cosmetic allergen, or a latex allergen. In some embodiments, the antibody is an antibody that specifically binds to a food allergen, such as a milk allergen, an egg allergen, a nut allergen, a fish allergen, a shellfish allergen, a soy allergen, a legume allergen, a seed allergen, or a wheat allergen. In some embodiments, the antibody specifically binds to a peanut allergen.

IgG antibodies binds pathogens, toxins, and is associated with Type II and II hypersensitivity reactions. IgG also activates the complement system of the immune system, helping to clear pathogens and damaged cells, and plays a role in the inflammatory cascade. By binding to the epitope, or binding region, on allergens or pathogens, IgG helps to alert the immune system to the presence of the foreign object and activate the immune system to clear it from the body.

IgE antibodies mediate Type I hypersensitivity reactions by binding to specific receptors on inflammatory immune cells, such as mast cells in mucosal tissues lining body surfaces and cavities, as well as basophils in the circulation. Those cells mediate allergic responses triggered 20 by specific antigens (allergens) that are recognized by IgE through the release of inflammatory molecules, such as histamine. The inflammatory response leads to symptoms, such as sneezing, runny or stuffed nose, itchy eyes, breathing difficulties, and, in extreme cases, anaphylactic shock and even death.

The FcεRI receptor is also known as the high-affinity IgE receptor. It is present on mast cells, basophils, eosinophils, and epidermal Langerhans cells. FcεRI is a known promoter of histamine, among other immune mediators, and therefore can activate the inflammatory cascade. Blockade of high affinity FcεRI receptors is likely to slow the timing of and reduce the severity of the inflammatory response.

According to an embodiment of the invention, the anti-IgE antibody is omalizumab. Omalizumab is a recombinant DNA-derived humanized IgG1_{K} monoclonal antibody that selectively binds to human immunoglobulin E (IgE). The antibody has a molecular weight of approximately 149 kiloDaltons. Omalizumab is an anti-IgE antibody indicated for: moderate to severe persistent asthma in adults and pediatric patients 6 years of age and older with a positive skin test or in vitro reactivity to a perennial aeroallergen and symptoms that are inadequately controlled with inhaled corticosteroids; nasal polyps in adult patients 18 years of age and older with inadequate response to nasal corticosteroids, as add-on maintenance treatment; and chronic spontaneous urticaria (CSU) in adults and adolescents 12 years of age and older who remain symptomatic despite H1 antihistamine treatment.

According to varying embodiments of the invention, the anti-IgE antibody reduces circulating IgE by at least 25%, by at least 50%, or by at least 75%. The goal of treating a subject with the composition is to reduce serum IgE levels so that a Type I hypersensitivity reaction is not as sudden or severe, allowing the subject more time before lifesaving emergency treatment can be provided.

Embodiments of the invention further provide a method of treating a condition in a subject, the method comprising providing to a subject the composition for allergy treatment comprising an IgG antibody that binds to an epitope on an allergen and an anti-IgE antibody in an amount sufficient to inhibit IgE binding to a high affinity FcεRI receptor but that does not eliminate circulating IgE. The method also comprises any of the varied embodiments of the composition discussed above.

In aspects of the invention, the antigen-binding function of an antibody or fragment of the invention may be performed by fragments of the full-length antibody comprising the binding portion of the antibody. Examples of binding portions of antibodies include a monovalent fragment consisting of the variable light chain, variable heavy chain, and CL and CH1 constant regions (Fab fragment). Examples also include bivalent fragments comprising two Fab fragments linked by a disulfide bridge at the hinge region (F(ab′)2 fragment), fragments consisting of the variable heavy chain and CH1 constant domain (Fd fragment), fragments consisting of the variable light chain and variable heavy chain domains of a single arm of an antibody (Fv fragment), fragments consisting of a variable heavy chain domain (dAb fragment), and isolated complementarity determining region.

According to varying embodiments of the invention, the IgG antibody and anti-IgE antibody are contained within one dosage form, whereas in another embodiment, the IgG antibody and anti-IgE antibody are provided to the subject in separate dosage forms. In some embodiments, antibodies of the invention are delivered directly in a prolonged release formulation. The antibody itself may be modified to include features that increase serum half-life. Antibodies may be pegylated, conjugated to other proteins (e.g., human serum albumin) or provided in a vehicle that causes delayed release of the antibody.

Embodiments of the present invention provide for the administration of a therapeutically effective amount of a pharmaceutical formulation to a subject for preventing or treating an allergic response in said subject. The formulation generally includes a composition comprising the vector and other components, such as, for example, one or more pharmaceutically acceptable carriers, adjuvants, and/or vehicles appropriate for the particular route of administration for which the composition is to be employed. In some embodiments, the carrier, adjuvant, and/or vehicle is suitable for injection (via a needle of the like) for intravenous, intramuscular, intraperitoneal, transdermal, or subcutaneous administration, as well as a consumable, or spray for related oral and inhalant administrations.

Therapeutic compositions of the invention may comprise an antibody, or antigen-binding portion thereof, formulated for delivery. Delivery may be in oral, subcutaneous, intravenous, aerosol or other appropriate formulations. Alternatively, therapeutic compositions of the invention may be delivered in the form of a nucleic acid encoding an appropriate antibody or antigen-binding portion thereof.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A composition for allergy treatment, the composition comprising:
an IgG antibody that binds to an epitope on an allergen; and
an anti-IgE antibody in an amount sufficient to inhibit IgE binding to a high affinity FcεRI receptor but that does not eliminate circulating IgE.

2. The composition of claim 1, wherein the allergy treated is a hypersensitivity reaction.

3. The composition of claim 1, wherein the allergy treated is an anaphylactoid Type I hypersensitivity reaction.

4. The composition of claim 1, wherein the allergy treated is anaphylaxis due to exposure to a food, animal, or environmental allergen.

5. The composition of claim 1, wherein the anti-IgE antibody is omalizumab.

6. The composition of claim 1, wherein the anti-IgE antibody reduces circulating IgE by at least 25%.

7. The composition of claim 1, wherein the anti-IgE antibody reduces circulating IgE by at least 50%.

8. The composition of claim 1, wherein the anti-IgE antibody reduces circulating IgE by at least 75%.

9. The composition of claim 1, wherein the IgG antibody and anti-IgE antibody are contained within one dosage form.

10. The composition of claim 1, wherein the IgG antibody and anti-IgE antibody are provided to the subject in separate dosage forms.

11. A method of treating a condition in a subject, the method comprising
providing to a subject a composition for allergy treatment comprising an IgG antibody that binds to an epitope on an allergen; and an anti-IgE antibody in an amount sufficient to inhibit IgE binding to a high affinity FcεRI receptor but that does not eliminate circulating IgE.

12. The method of claim 11, wherein the allergy treated is a hypersensitivity reaction.

13. The method of claim 11, wherein the allergy treated is an anaphylactoid Type I hypersensitivity reaction.

14. The method of claim 11, wherein the allergy treated is anaphylaxis due to exposure to a food, animal, or environmental allergen.

15. The method of claim 11, wherein the anti-IgE antibody is omalizumab.

16. The method of claim 11, wherein the anti-IgE antibody reduces circulating IgE by at least 25%.

17. The method of claim 11, wherein the anti-IgE antibody reduces circulating IgE by at least 50%.

18. The method of claim 11, wherein the anti-IgE antibody reduces circulating IgE by at least 75%.

19. The method of claim 11, wherein the IgG antibody and anti-IgE antibody are contained within one dosage form.

20. The method of claim 11, wherein the IgG antibody and anti-IgE antibody are provided to the subject in separate dosage forms.
